(19) United States
(12) Patent Application Publication
(10) Pub. No.: US 2012/0172910 A1
(43) Pub. Date: Jul. 5, 2012

(54) SMART EXPANDABLE MEMBER FOR MEDICAL APPLICATIONS

(76) Inventors: David C. Forster, Los Altos Hills, CA (US); Yenyu Chen, Palo Alto, CA (US)

(21) Appl. No.: 12/981,285

(22) Filed: Dec. 29, 2010

(51) Int. Cl. A61M 29/00

(52) U.S. Cl. 606/194

(57) ABSTRACT

Devices and methods for assessing the compliance of vessel lumens and hollow portions of organs are described. The devices and methods are particularly adapted for determining the compliance of the native heart valves to facilitate the later implantation of a prosthetic heart valve. The devices are typically catheter-based having an expandable member fixed to a distal end of the catheter. Located within the expandable member is an imaging member. The methods typically comprise deploying the balloon percutaneously to a target location, expanding the balloon, and determining the compliance of a lumen, particularly a cardiac valve. An optical coherence tomography apparatus is a preferred apparatus for determining compliance.

### FIELD OF THE INVENTION

The present invention relates generally to medical devices and methods. More particularly, the present invention relates to methods and devices for assessing the compliance of lumens and surrounding tissue. The devices and methods are particularly adapted for use during minimally invasive surgical interventions, but may also find application during surgical replacement on a stopped heart, less invasive surgical procedures on a beating heart, and other percutaneous procedures.

### BACKGROUND OF THE INVENTION

Minimally invasive surgery provides several advantages over conventional surgical procedures, including reduced recovery time, reduced surgically-induced trauma, and reduced post-surgical pain. Moreover, the expertise of surgeons performing minimally invasive surgery has increased significantly since the introduction of such techniques in the 1980s. As a result, substantial focus has been paid over the past twenty years to devices and methods for facilitating and improving minimally invasive surgical procedures.

One area in which there remains a need for substantial improvement is pre-surgical assessment of treatment locations intended to be subjected to a minimally invasive surgical procedure. For example, when a surgical procedure is to be performed at a treatment location within the body of a patient, it would frequently be beneficial for the surgeon to have prior knowledge of the compliance of the treatment location. This information would be particularly useful in relation to minimally invasive surgical procedures in which prosthetic devices are implanted within a body lumen or within a hollow portion of an organ located within the body of the patient. Such information could then be used to select the size and/or shape of the prosthetic device to more closely match the size, shape, and topography of the treatment location.

A particular portion of the anatomy for which complete and accurate physical assessment would be beneficial are the coronary valves. Diseases and other disorders of heart valves affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation, permitting backflow of blood in the heart.

Medication may be used to treat some heart valve disorders, but many cases require replacement of the native valve with a prosthetic heart valve. In such cases, a thorough assessment of the compliance of the native valve annulus would be extremely beneficial. Prosthetic heart valves can be used to replace any of the native heart valves (aortic, mitral, tricuspid or pulmonary), although repair or replacement of the aortic or mitral valves is most common because they reside in the left side of the heart where pressures are the greatest.

A conventional heart valve replacement surgery involves accessing the heart in the patent's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. After the heart has been arrested the aorta is cut open to allow access to the diseased valve for replacement. Such open heart surgery is particularly invasive and involves a lengthy and difficult recovery period.

Less invasive approaches to valve replacement have been proposed. The percutaneous implantation of a prosthetic valve is a preferred procedure because the operation is performed under local anesthesia, does not require cardiopulmonary bypass, and is less traumatic.

### BRIEF SUMMARY OF THE INVENTION

The present invention provides methods and devices for assessing the compliance of a vessel lumen or a hollow portion of an organ located within a patient. The methods and devices may find use in the coronary vasculature, the atrial appendage, the peripheral vasculature, the abdominal vasculature, and in other ducts such as the biliary duct, the fallopian tubes, and similar lumen structures within the body of a patient. The methods and devices may also find use in the heart, lungs, kidneys, or other organs within the body of a patient. Moreover, although particularly adapted for use in vessels and organs found in the human body, the apparatus and methods may also find application in the treatment of animals.

However, the primary use of the methods and devices described herein is in the assessment of the compliance of the native heart valves. Such assessment of compliance is useful to facilitate proper orientation, sizing, selection, and implantation of prosthetic heart valves into the native valve space. Proper orientation, selection and sizing ensures that the prosthetic heart valve that is delivered during the implantation procedure will be of a size and shape that fits within the native valve space, including accommodations for any defects or deformities that are detected by the assessment process. Proper orientation, selection and sizing also ensures that the prosthetic valve, once fully expanded, will properly seal against the aortic wall to prevent leakage, and to prevent migration of the prosthetic valve.

The methods and devices described herein are suitable for use in facilitating the orientation, selection and sizing of prosthetic heart valves of all types, independent of the design, implantation mechanism, deployment technique, or any other aspect of the prosthetic valve. In many cases, particularly in the case of a prosthetic valve that is expandable from a delivery state to a deployed state, the assessment of the native valve space is of very great importance. For example, it is important to know the compliance of the native valve space when the valve space has been placed under the expansive load that is produced by the prosthetic valve. If the valve does not fit properly, it may migrate, leak, or resist deployment altogether.

The methods include use of an assessment member that is preferably located at or near the distal end of a catheter or other similar device. The assessment member is introduced to a treatment location within the patient, preferably the native cardiac valve, where the assessment member is activated or otherwise put into use to perform an assessment of the compliance of the treatment location, to collect the assessment information, and to provide the assessment information to the clinician. The compliance of the lumen or hollow portion of the organ is a central aspect of the present invention. Compliance is a measure of the lumen or hollow portion of the organ and can be defined as the rate of change of a physical property of the lumen or hollow portion of the organ with a change in force that causes change of the physical property.

Additional assessment information may be gathered during the assessment of a physical property and may include the size (e.g., diameter, circumference, area, volume, etc.) of the valve space, the shape (e.g., round, spherical, irregular, etc.) of the lumen or hollow portion of the organ, the topography (e.g., locations, sizes, and shapes of any irregular features) of the lumen or hollow portion of the organ, the nature of any regular or irregular features (e.g., thrombosis, calcification, healthy tissue, fibrosa), the spatial orientation (e.g., absolute location relative to a fixed reference point, or directional orientation) of a point or other portion of the treatment location, or the thickness, density, reflectivity, and other physical properties of the lumen or hollow portion of the organ.

Access to the treatment location is obtained by any conventional method, such as by general surgical techniques, less invasive surgical techniques, or percutaneously. A preferred method of accessing the treatment location is transluminally, preferably by well-known techniques for accessing the vasculature from a location such as the femoral artery. The catheter is preferably adapted to engage and track over a guidewire that has been previously inserted and routed to the treatment site.

The assessment mechanism includes an expandable member that is attached to the catheter shaft at or near its distal end. The expandable member may comprise an inflatable balloon or balloon-like member having limited elasticity, a structure containing a plurality of interconnected metallic or polymeric springs or struts, an expandable “wisk”-like structure, or other suitable expandable member. In the case of an inflatable balloon, the expandable member is operatively connected to a source of inflation medium that is accessible at or near the proximal end of the catheter. The expandable member has at least two states, an unexpanded or contracted state and an expanded state. The unexpanded state generally corresponds with delivery of the assessment mechanism through the patient's vasculature. The expanded state generally corresponds with the assessment process. It is understood that assessment process may continue during contraction of the expandable member. The expandable member is adapted to provide assessment information to the user when the expandable member is engaged with a treatment location within the body of a patient.

Turning to several exemplary devices and methods, in one aspect of the invention, a catheter-based system includes a transluminal imaging device contained partially or entirely within an expandable structure attached at or near the distal end of the catheter.

In an exemplary embodiment, the imaging device may be an ultrasonic imaging probe that is configured to transmit and receive ultrasonic signals at a desired frequency or at a plurality of desired frequencies. The received signals are then used to locate an outer periphery of the expandable member with respect to the shape and orientation of the lumen or hollow portion of the organ. In other exemplary embodiments, the imaging device may be an optical imaging device or an acoustic imaging device. It is understood that other imaging devices not discussed in detail that measure a physical property are within the scope of the invention.

In the preferred embodiments, the expandable member is a balloon member. The balloon member is connected to an inflation lumen that runs between the proximal and distal ends of the catheter, and that is selectively attached to a source of inflation medium at or near the proximal end of the catheter. The balloon member is thereby selectively expandable while the imaging device is located either partially or entirely within the interior of the balloon. The imaging device is adapted to be advanced, retracted, and rotated within the balloon, thereby providing for imaging in a plurality of planes and providing the ability to produce three-dimensional images of the treatment site.

In optional embodiments, the expandable member is filled with a medium that enhances the imaging process. For example, the medium may comprise a material that increases the transmission capabilities of the ultrasonic waves, or that reduces the amount of scattering of the ultrasonic waves that would otherwise occur without use of the imaging-enhancing medium. In still other optional embodiments, the expandable structure contains (e.g., has embedded or formed within) or is formed of a material that enhances the imaging process. In still other embodiments, the expandable member includes a layer of or is coated with a material that enhances the imaging process.

In use, the transluminal imaging device is first introduced to the target location within the patient, such as the native valve annulus. In the preferred embodiment, this is achieved by introducing the catheter through the patient's vasculature to the target location. Typically, the catheter tracks over a guidewire that has been previously installed in any suitable manner. The imaging device may be provided with a radiopaque or other suitable marker at or near its distal end in order to facilitate delivery of the imaging device to the target location by fluoroscopic visualization or other suitable means. Once the imaging device is properly located at the target location, the expandable structure is expanded by introducing an expansion medium through the catheter lumen. The expandable structure expands such that it engages and applies pressure to the internal walls of the target location, such as the valve annulus. The expandable structure also takes on the shape of the internal surface of the target location, including all contours or other topography. Once the expandable structure has been sufficiently expanded, the imaging device is activated. Where appropriate, the imaging device is advanced, retracted, and/or rotated to provide sufficient movement to allow a suitable image of the target location to be created, or to collect a desired amount of measurement information. The measurement information collected and/or the images created by the imaging device are then transmitted to a suitable user interface, where they are displayed to the clinician.

In use, the expandable member is first introduced to the target location within the patient. In the preferred embodiment, this is achieved by introducing the catheter through the patient's vasculature to the target location. The catheter tracks over a guidewire that has been previously installed in any suitable manner. The expandable member carried on the catheter may be provided with a radiopaque or other suitable marker at or near its distal end in order to facilitate delivery of the physical assessment member to the target location by fluoroscopic visualization or other suitable means. Once the expandable member is properly located at the target location, the expandable member is expanded by introducing an expansion medium through the catheter lumen. The expandable member expands to a size such that the expandable member is able to engage and expand the lumen or hollow portion of the organ, thereby providing an indicator of the compliance of the lumen or hollow portion of the organ. In this way, the clinician is able to obtain precise measurements of the compliance of the lumen or hollow portion of the organ at the target location.

The present invention further includes an optical coherence tomography (OCT) apparatus for determining a physical property within a lumen or hollow portion of an organ. The OCT apparatus includes, in one exemplary embodiment, a dual stage mirror and in another exemplary embodiment, a single stage mirror, which enables a field of view much greater than has been done before with conventional OCT apparatus. That is, typical OCT apparatus can only move within a distance of about 2 millimeters (mm). It would be desirable to move over longer distances, such as 0 to 20 mm, so as to be able to determine physical properties within a lumen or hollow portion of an organ. The OCT apparatus has particular applicability for determining physical properties such as the dimensions useful to understand compliance.

In a further aspect of the present invention, a valvuloplasty procedure is performed in association with the assessment of the native cardiac valve. In a first embodiment, the expandable member also functions as a valvuloplasty balloon. The expandable member is placed within the cardiac valve space, where it is expanded. Expansion of the expandable member causes the native valve to increase in size and forces the valve, which is typically in a diseased state in which it is stiff and decreased in diameter, to open more broadly. The valvuloplasty procedure may therefore be performed prior to the deployment of a prosthetic valve, but during a single interventional procedure. In a further preferred embodiment, the expandable member after performing valvuloplasty may be expanded beyond the shape and size of the native cardiac valve to distort the native cardiac valve and perform an assessment function.

The measurement and diagnostic processes performed by any of the foregoing devices and methods may be used to facilitate any suitable medical diagnosis, treatment, or other therapeutic processes. One particular treatment that is facilitated by the foregoing devices and methods is the repair and/or replacement of coronary valves, particularly aortic valve replacement using a prosthetic valve.

Other aspects, features, and functions of the inventions described herein will become apparent by reference to the drawings and the detailed description of the preferred embodiments set forth below.

### DESCRIPTION OF THE DRAWINGS

FIG. 1 is a perspective view of a catheter in accordance with several of the embodiments of the present invention.

FIG. 2A is a cross-sectional view of an imaging device in accordance with the present invention.

FIG. 2B is a cross-sectional view of the imaging device of FIG. 2A, showing an expandable member in its expanded state.

FIG. 3 is an illustration of an exemplary apparatus for performing expansion of the expandable member to determine compliance.

FIG. 4A is a cross section of a lumen showing an area of calcification.

FIG. 4B is a cross sections of a lumen showing a lumen at two different points in time.

FIG. 5 is a graph of dimension of a lumen or hollow portion of an organ plotted versus pressure in the expandable member.

FIG. 6 is a graph of pressure versus time according to an exemplary embodiment.

FIG. 7 is a graph of pressure versus time according to a second exemplary embodiment.

FIG. 8 is a graph of pressure versus time according to a third exemplary embodiment.

FIG. 9A is a graph of dimension of a lumen plotted versus pressure during a valvuloplasty procedure.

FIG. 9B is a graph showing dilation and return of a lumen during a valvuloplasty procedure.

FIG. 10 is a schematic diagram of an optical coherence tomography apparatus for measuring the dimensions of the expansion of the expandable member.

FIG. 11 is a perspective view of a dual stage mirror which forms a part of the optical coherence tomography apparatus of FIG. 10.

FIG. 12 is a cross sectional view of an exemplary embodiment of a dual stage mirror which may form a part of the optical coherent tomography apparatus of FIG. 10.

FIG. 13 is a cross sectional view of an exemplary embodiment of a single stage mirror which may form a part of the optical coherent tomography apparatus of FIG. 10.

FIG. 14 is a schematic of a control system having a feedback loop for controlling the translation stage of the optical coherence tomography apparatus.

### DETAILED DESCRIPTION OF THE PREFERRED EMBODIMENTS

The present invention is directed to methods and devices for assessing the compliance of anatomical vessels and organs using minimally invasive surgical techniques. As summarized above, the devices are typically catheter-based devices. Such devices are suitable for use during less invasive and minimally invasive surgical procedures. However, it should be understood that the devices and methods described herein are also suitable for use during surgical procedures that are more invasive than the preferred minimally invasive techniques described herein.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

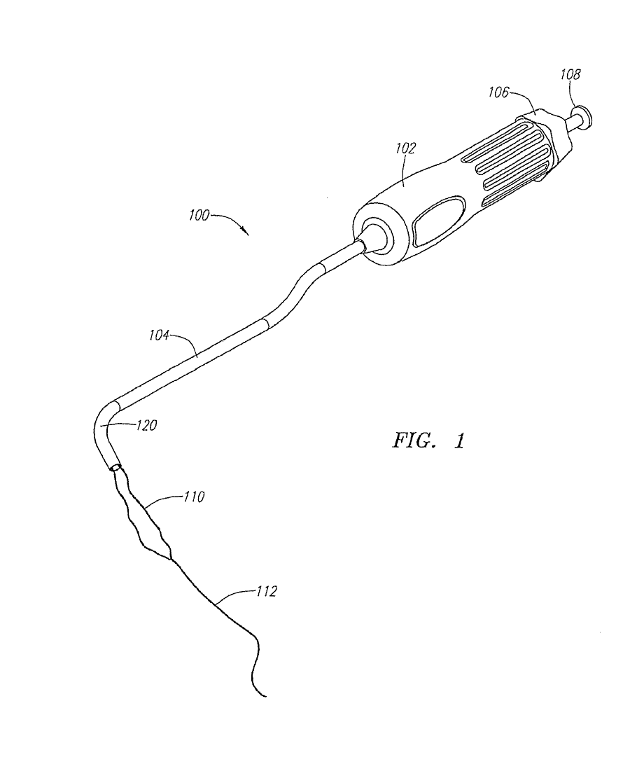

Turning to the drawings, FIG. 1 shows a catheter 100 suitable for use with each of the assessment mechanisms described herein. The catheter 100 includes a handle 102 attached to the proximal end of an elongated catheter shaft 104. The size and shape of the handle 102 may vary, as may the features and functionality provided by the handle 102. In the illustrated embodiment, the handle 102 includes a knob 106 rotatably attached to the proximal end of the handle 102. The knob 106 may be rotated to control the movement and/or function of one or more components associated with the catheter 100, such as for retraction of one or more catheter shafts or sheaths, or manipulation of an expandable member or other component carried at or near the distal end of the catheter shaft 104. Alternative structures may be substituted for the knob 106, such as one or more sliders, ratchet mechanisms, or other suitable control mechanisms known to those skilled in the art.

An inflation port 108 is located near the proximal end of the handle 102. The inflation port 108 is operatively connected to at least one inflation lumen that extends through the catheter shaft 104 to an expandable member 110 located near the distal end of the catheter shaft 104. The inflation port 108 is of any suitable type known to those skilled in the art for engaging an appropriate mechanism for providing an inflation medium to inflate the expandable member 110. For example, a suitable inflation mechanism is an Indeflator™ inflation device, manufactured by Guidant Corporation, or a medical syringe can be used.

The catheter 100 is adapted to track a guidewire 112 that has been previously implanted into a patient and routed to an appropriate treatment location. A guidewire lumen extends through at least the distal portion of the catheter shaft 104, thereby providing the catheter 100 with the ability to track the guidewire 112 to the treatment location. The catheter 100 may be provided with an over-the-wire construction, in which case the guidewire lumen extends through the entire length of the device. Alternatively, the catheter 100 may be provided with a rapid-exchange feature, in which case the guidewire lumen exits the catheter shaft 104 through an exit port at a point nearer to the distal end of the catheter shaft 104 than the proximal end thereof.

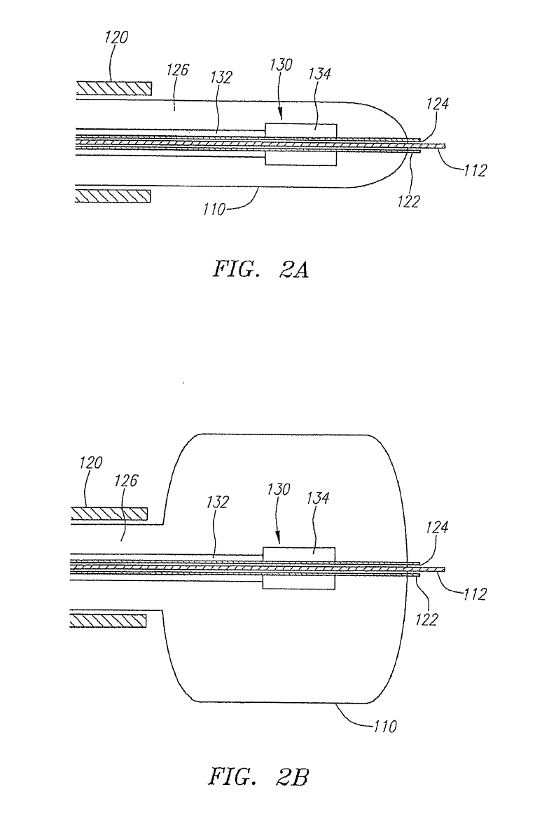

Turning next to FIGS. 2A-B, an assessment mechanism is shown and described. The assessment mechanism is located at the distal end of a catheter 100, such as that illustrated in FIG. 1 and described above. The assessment mechanism shown in FIGS. 2A-B includes an imaging device that is used to provide two-dimensional or three-dimensional images of a vessel lumen or the hollow portion of an organ within the body of a patient, as described below.

The assessment mechanism includes the outer sheath 120 of the catheter shaft 104, which surrounds the expandable member 110. In the preferred embodiment, the expandable member 110 is an inflatable balloon. The expandable member 110 is attached at its distal end to a guidewire shaft 122, which defines a guidewire lumen 124 therethrough. The guidewire 112 extends through the guidewire lumen 124.

An imaging member 130 is contained within the expandable member 110. The imaging member 130 is supported by a shaft 132 that extends proximally to the handle 102, where it is independently controlled by the user. The imaging member shaft 132 is coaxial with and surrounds the guidewire shaft 124, but is preferably movable (e.g., by sliding) independently of the guidewire shaft 124. At the distal end of the imaging member shaft 132 is the imaging head 134. The imaging head 134 may be any mechanism suitable for transmitting and receiving ultrasonic waves. In a preferred embodiment, there may be a plurality of imaging heads 134, although only one such imaging head 134 is shown for clarity. A typical imaging head 134 is an ultrasonic imaging probe. It is within the scope of the present invention to have other imaging members 130. Such other imaging members 130 may include but not be limited to an optical fiber in conjunction with optical coherence tomography for optical imaging or an acoustic imaging device for transesophageal echo.

In an alternate embodiment (not shown) the imaging member could be integrated into the guidewire to form a single unit.

The expandable member 110 is subject to expansion when a suitable expansion medium is injected into the expandable member through the inflation lumen 126. The inflation lumen 126, in turn, is connected to the inflation port 108 associated with the handle 102. FIG. 2A illustrates the expandable member 110 in its unexpanded (contracted) state, while FIG. 2B illustrates the expandable member 110 in its expanded state, such as after a suitable inflation medium is injected through the inflation port 108 and inflation lumen 126 into the expandable member 110.

To use the assessment mechanism illustrated in FIGS. 2A-B, the distal portion of the catheter is delivered to a treatment location within the body of a patient over the previously deployed guidewire 112. In a particularly preferred embodiment, the treatment location is the aortic heart valve, and the guidewire 112 is deployed through the patient's vasculature from an entry point in the femoral artery using, for example, the Seldinger technique. Deployment of the assessment mechanism is preferably monitored using fluoroscopy or other suitable visualization mechanism. Upon encountering the treatment location, the expandable member 110 is expanded by inflating the balloon with a suitable inflation medium through the inflation port 108 and the inflation lumen 126. The expandable member 110 engages the internal surfaces of the treatment location, such as the annular root of the aortic heart valve. In the case of a valvuloplasty procedure, the inflatable member is further dialated after initial contact with the anatomy until a stenotic valve is dialated to an increased valvular area. The expandable member 110 is expanded, the imaging member 130 is activated and the imaging process is initiated. The imaging member 130 is preferably advanced, retracted, and rotated within the expandable member 110 as needed to obtain images in a variety of planes to yield a 360° three-dimensional image, or any desired portion thereof. The activation of the imaging member may be present during any portion or all of the expansion phase, the dilation phase, and/or the contraction/deflation phase. Once the imaging process is completed, the expandable member 110 is fully deflated, and the assessment mechanism may be retracted within the catheter shaft 104. The catheter 100 is then removed from the patient. Alternately, the assessment mechanism and catheter 100 can be removed simultaneously.

Optionally, the inflation medium used to expand the expandable member 110 may comprise a material that enhances the ability of the imaging member 130 to generate images. For example, the inflation medium may facilitate enhanced acoustic transmission, reception, or it may reduce the incidence of scattering of the assessment signal. Such suitable inflation media may include a liquid or a gas and more specifically may include, for example, the following: acoustic gel, dielectric fluid, saline, blood, gas, contrast medium and the like. These effects may be enhanced further by provision of a material or coating on the surface of the expandable member 110 that optimizes the imaging process. Such suitable materials and/or coatings include relatively dense materials such as metal, ceramic, high density polymers, and the like.

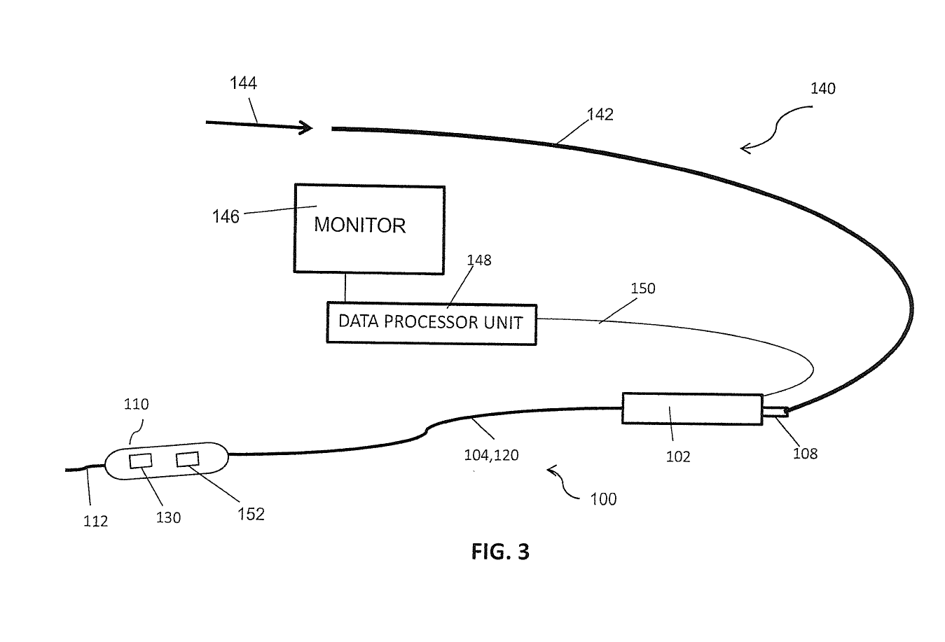

Referring now to FIG. 3, there is shown an inflation apparatus 140 for expansion of an expandable member during a procedure to determine physical properties of a structure including but not limited to size, shape, or compliance. As described in FIG. 1, there is a catheter 100 which includes a handle 102, catheter shaft 104, outer sheath 120, expandable member 110 and guide wire 112. The handle 102 has an inflation port 108.

The inflation apparatus 140 includes a tube 142 for carrying an inflation medium (not shown) to the catheter 100. The inflation medium may be provided to the tube 142 at an end thereof as indicated by arrow 144. The inflation medium flows through the tube 142 and catheter 100, eventually ending up in expandable member 110 to cause the expandable member 110 to expand. The pressure of the inflation medium may be sensed anywhere in the pressurized system from the introduction of the inflation medium to the expandable member. Conventional pressure sensors (not shown) may be incorporated accordingly and operatively connected to the instrumentality for receiving data. For purposes of illustration and not limitation, pressure sensor 152 is shown within expandable member 110. Monitoring the expansion of the expandable member 110 is an instrumentality for receiving, recording, processing, communicating, displaying, and/or printing assessment information. For purposes of illustration and not limitation, the instrumentality in an exemplary embodiment may be a data processor unit 148.

Data processor unit 148 may be a computer, computer processor or microprocessor and may include random access memory (RAM), read-only memory (ROM) and a storage device of some type such as a hard disk drive, floppy disk drive, CD-ROM drive, tape drive or other storage device. Data processor unit 148 may also include communication links to provide communication to other devices such as another computer. Data processor unit 148 may be linked to a monitor 146, which may also be a computer such as a laptop, so that the clinician may view the expansion of the expandable member and also control the expansion manually if desired.

Catheter 100 may also include an assessment mechanism as described previously. One such assessment mechanism is an imaging member 130 as described previously which may assist in determining the compliance of the lumen or hollow portion of an organ in real time. Other assessment mechanisms may be present such as pressure sensors (for example, a pressure transducer) to measure the pressure in the expandable member 110.

The assessment mechanisms provide feedback to data processor unit 148. For this purpose, wire 150 extends from handle 102 of the catheter 100 to the data processor unit 148. Wire 150 actually extends up into expandable member 110 to relay information from imaging member 130 and pressure sensor 152 to data processor unit 148. It is within the scope of the present invention for imaging member 130 and pressure sensor 152 to communicate wirelessly with data processor unit 148.

Based on the feedback provided to data processor unit 148 from the assessment mechanisms, compliance of the lumen or hollow portion of an organ may be determined. Compliance may be useful in varying the rate and the extent of expansion and deflation of the expandable member 110.

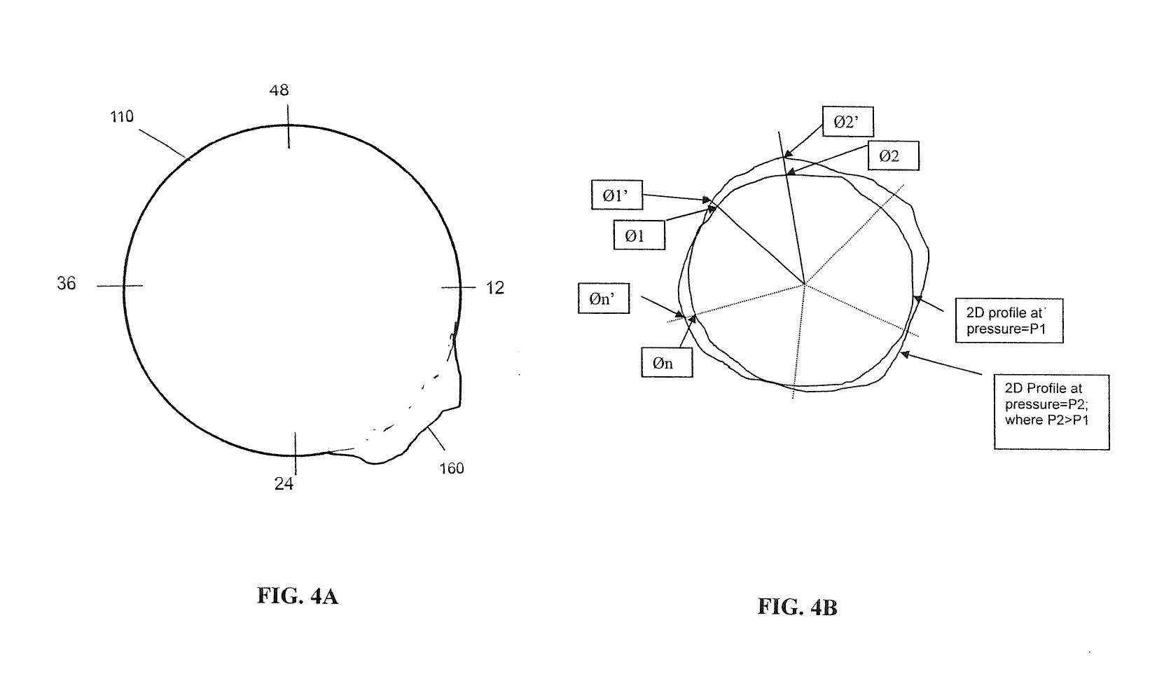

Referring now to FIG. 4A, a 2D cross section of an expandable member 110 impinging against a wall of a lumen (not shown) is illustrated. The expandable member 110 may have an irregular circumference 160 as it impinges against a diseased portion of the lumen. It is desired to be able to determine physical properties of a structure including but not limited to size, shape, and compliance of the lumen. The expandable member 110 is expanded, the imaging member 130 shown in FIG. 3 is activated and the imaging process is initiated. The imaging member 130 is preferably advanced, retracted, and rotated within the expandable member 110 as needed to obtain images in a variety of planes to yield a 360° three-dimensional image, or any desired portion thereof. The imaging member 130 takes readings at multiple locations around the circumference of the expandable member 110 during various stages of either expansion, contraction, or both. According to an exemplary embodiment, the number of locations where readings are done is 48. There may be more than 48 locations or less than 48 locations in other exemplary embodiments as the number “48” of locations is not critical. FIG. 4A illustrates four of those locations. The diseased portion of the lumen is in the second quadrant of the expandable member 110 between locations 12 and 24. Given the example of 48 points, data for each of these 48 points is gathered at different points of expansion of the expandable member at different points in time. The presence of calcification, lesions or the like will have less change in the physical property of displacement over a specific change in pressure than healthy, non-calcified tissue. As the expandable member touches the lumen and continues to expand, each of these points may behave differently. Each of these points is monitored during either inflation of the expandable member in the case of a balloon, or the contraction/deflation, or both.

One of the possible physical properties measured can be the 2D or 3D profile of the lumen at different points in time and/or various pressures. FIG. 4B shows a 2D profile at Pressure 1 and a second 2D profile at Pressure 2 which is greater than Pressure 1. It can be seen that the distance between θ2 and θ2′ is greater than the distance between θ1 and θ1′ indicating more movement, greater compliance and, in the case of tissue, less disease. FIG. 4B shows θ1, θ2, through θn where n=48 in this example.

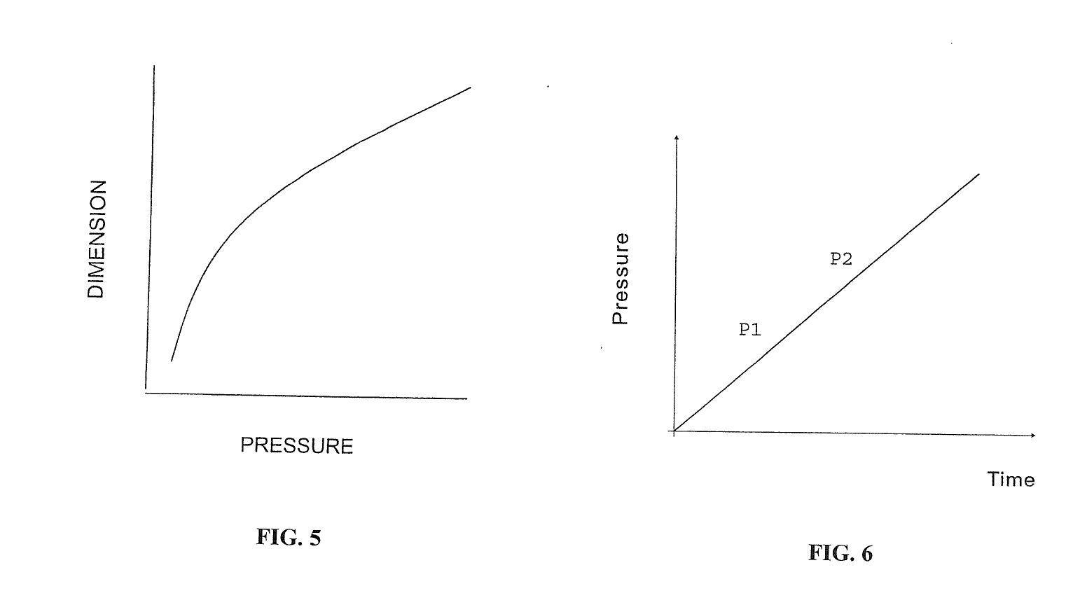

The imaging member 130 sends readings of waves of light reflected off of the expandable member and/or the lumen or hollow portion of the organ in which it is located to the instrumentality shown as a data processor 148. The waves of light are analyzed by the data processor in this embodiment to determine dimensions of the lumen as the expandable member 110 is expanded. It is understood that the waves of light may be used by the instrumentality to determine other physical properties such as thickness, density, reflectivity, and other parameters as discussed earlier. Separately and also simultaneously, a force that causes a change of physical property applied to or within the expandable member 110 is measured as the expandable member 110 expands. The dimension of the expandable member 110, such as its radius is plotted versus the force as indicated in FIG. 5 for one of the 48 locations sampled by the imaging member 130. As there are 48 locations measured by the imaging member 130, there may be 48 graphs similar to FIG. 5 in order to have a complete two dimensional picture of the expandable member 110 and the lumen. If the imaging member 130 is translated along the longitudinal axis of the expandable member 110, then a three dimensional picture of the expandable member 110 and the lumen may be obtained as well.

The compliance may be determined from the graph in FIG. 5. As stated earlier, compliance may be defined as the rate of change of a physical property of the lumen with a change in force that causes change in that physical property. For purpose of discussion, the physical property is a dimension of the lumen or expandable member. The physical properties of the lumen may be measured indirectly by measuring the expandable member 110. Compliance may be expressed as follows:

$$compliance = \frac{θ - {θ}^{′}}{F 1 - F 2}$$

wherein
θ=a physical property at a location of the expandable member at a first point in time;
θ′=a physical property at the same location of the expandable member at a second point in time;
F1=a force applied by the expandable member at the first point in time; and
F2=a force applied by the expandable member at the second point in time.

Force is an input that causes a change in a dimension of the expandable member. The force may be applied as a pulling force, a pushing force, or a pressure applied by a fluid. For purposes of illustration and not limitation, the remaining discuss of the exemplary embodiments will focus on pressure as the applied force.

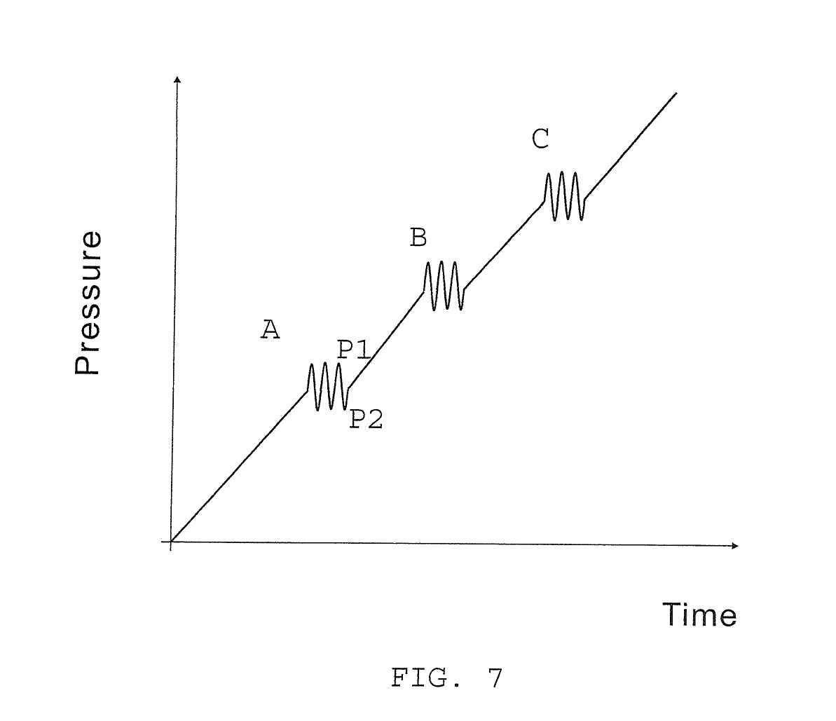

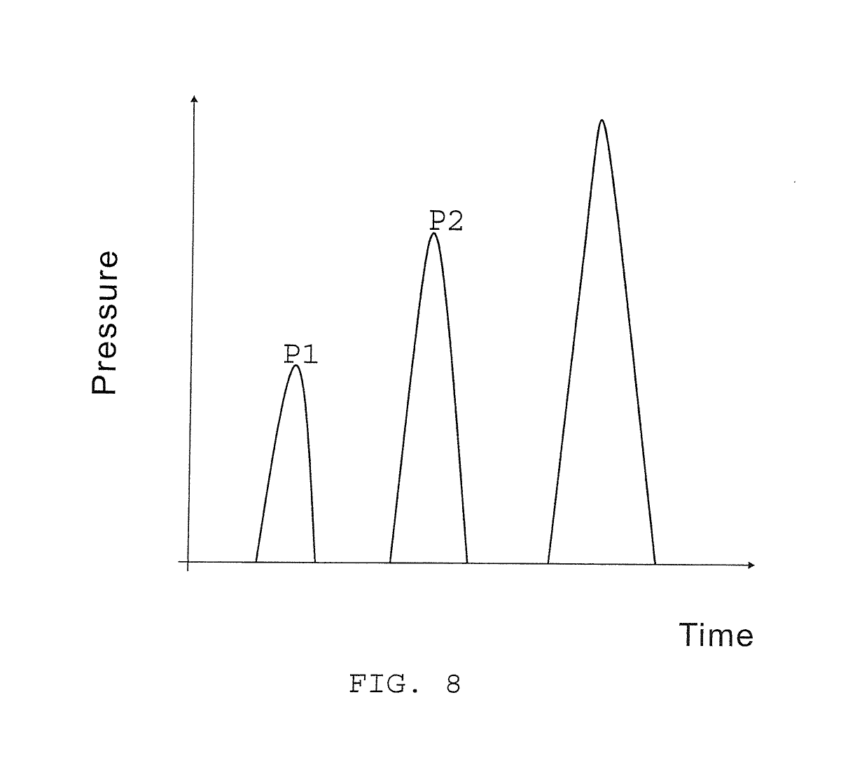

The dimensions of the lumen are determined from the dimensions of the expandable member 110 and are measured by the imaging member 130. Pressure may be determined any number of ways. In an exemplary embodiment, pressure is measured at a constant rate versus time as shown in FIG. 6. Pressure may be measured at any two points on the line at two different points in time and correlated with the change in dimensions at the same two points in time of the expandable member 110 (and hence the lumen also) to determine compliance of the lumen.

In another exemplary embodiment, the pressure may be pulsed as indicated in FIG. 7. During expansion of the expandable member 110, there is rapid inflation and deflation of the expandable member 110 at several locations, marked as A, B and C. Pulsing may occur at a pulse rate of about 1 to 1000 times per second. While pulsing, the pressure is increased and decreased by about ±0.1 atmospheres. During pulsing, the maximum pressure is held constant and the minimum pressure is held constant for each of the pulses so that during pulsing, the pulsing shifts the pressure versus time line horizontally. For purposes of determining compliance, P1 and P2 may be measured with each pulse as indicated in FIG. 7.

A further exemplary embodiment is illustrated in FIG. 8 which is a variation on pulsing. The pressure is increased and decreased a number of times and the maximum pressure increases with each pulse so that the pressure applied to the expandable member 110 is generally increasing upwardly during the expansion of the expandable member 110.

All of the data from the measurement of the dimensions of the expandable member 110 (and hence the lumen) and the pressure inside the expandable member 110 is sent to the data processing unit 148 for determining of the compliance of the lumen or hollow portion of an organ. All of the data, or selected parts of it, may be displayed on monitor 146 for review by the clinician who may take action as appropriate. There is a substantial quantity of data to be processed by the data processor unit 148 and it must be processed practically instantaneously so that the clinician has a real time view of the compliance of the lumen or hollow portion of an organ and can make an assessment of what is happening in the lumen or hollow portion of an organ at any point during the medical procedure.

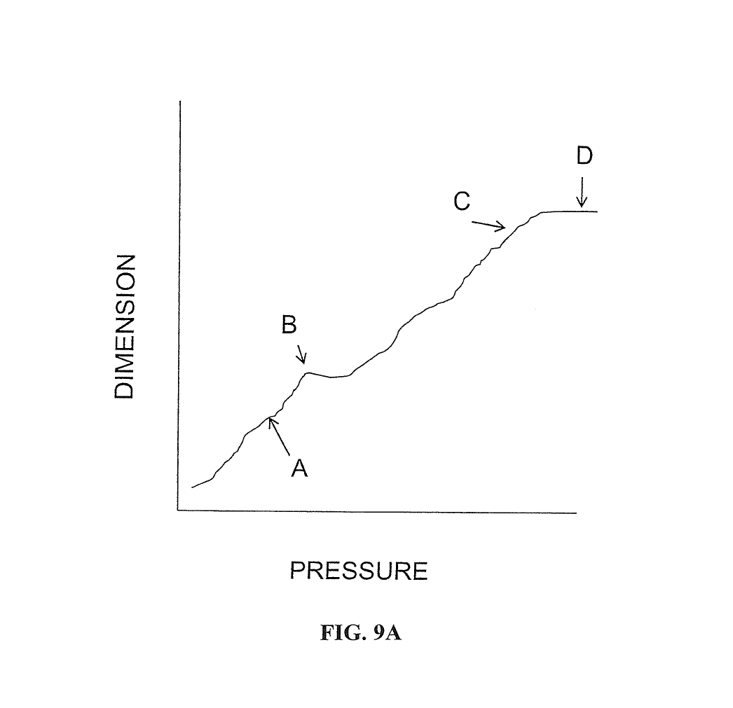

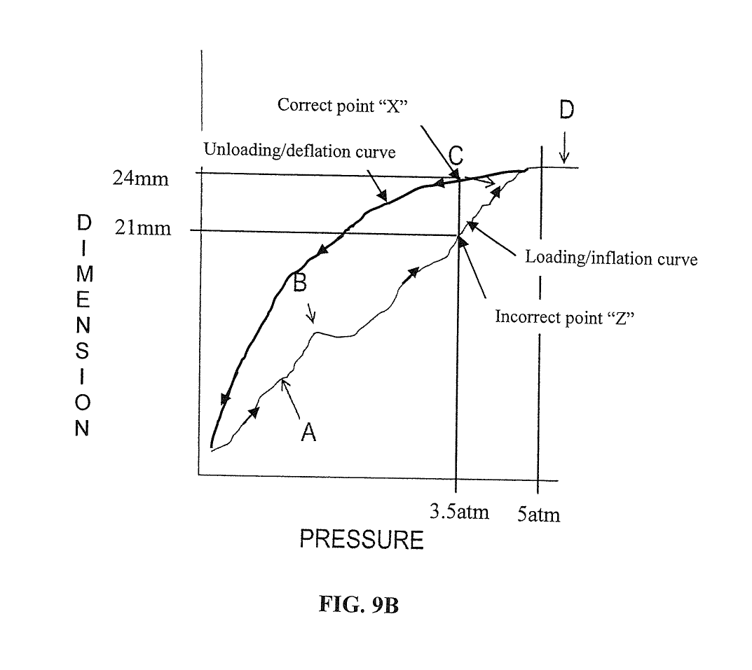

An hypothetical example may be formulated to illustrate the advantages of determining compliance for a valvuloplasty medical procedure. The following table and FIG. 9A illustrate results from an hypothetical valvuloplasty procedure.

| | Pressure | Dimension | | |
|---|---|---|---|---|
| Point on FIG. 9A | (atm) | (cm³) | Compliance | Action |
| A | <2.5 | <20 | >75% | Rapid expansion |
| B | <3 | <25 | <20% | Fracture |
| C | 3.5 | 35 | <30% | Slow down |
| D | >4 | >37 | <20% | Stop |

This example is based off of a valvuloplasty balloon that has a burst pressure of 5 atm and a volume at burst of 40 cm³. At point A, compliance has determined about 75% of the data points along a perimeter/circumference are moving easily. The low pressure and volume indicate that rapid expansion of the expandable member 110 may occur. At point B, of about 20% of the data points along a perimeter/circumference are moving easily and given the continued low volume and pressure, fracture of calcification is occurring. At point C, about 30% of the data points along a perimeter/circumference are moving easily, and given the volume is getting high and pressure is increasing this indicates that expansion of the lumen is nearing its upper limit and expansion of the expandable member 110 should slow down. Lastly, at point D, there is increase in pressure without much increase in volume and compliance is less than about 20%, indicating that the lumen is at its upper limit of expansion and therefore expansion of the expandable member 110 should stop and deflation should then occur before tearing of the lumen may occur.

The graph of FIG. 9A is not smooth because as valvuloplasty is performed and pressures are increased to enlarge a lumen, calcifications and lesions are fractured or disrupted resulting in rapid changes in dimension/volume and pressure.

Referring to FIG. 9B, the graph of FIG. 9A is repeated with the addition of data taken while the balloon is contracted as shown in the unloading/deflating curve. This curve shows that the inflation of the valvuloplasty balloon did work on the tissue by fracturing or disrupting calcification or other lesions resulting in a smoother profile. The area within the loading curve and unloading curve is hysteresis which is the work done to the tissue. The resultant tissue after valvuloplasty has different physical properties than before valvuloplasty due to the fracturing of the calcifications, and dilation of the lumen back to near normal diameter. The unloading/deflation curve shows that the lumen has a smoother curve and that the forces at a given pressure are reduced. The force to attain a given diameter after valvuloplasty is less than during valvuloplasty, because of the fracturing of calcification and resolution of lesions. It is important to obtain the properties of the tissue after modification by valvuloplasty, as it is this state that any further intervention or therapy such as implantation of a percutaneous valvular prosthesis would be subject to. For any given valvular prosthesis there is s corresponding structural strength for example radial strength of the valve frame. FIG. 9B shows an example in which a prosthetic valve frame has a structural strength equivalent to 3.5 atm. As can be seen in FIG. 9B there are two different valvular dimension results at 3.5 atm. One result is as the valvuloplasty is being performed which shows the corresponding valve size to be 21 mm. A second point on the unloading/deflation curve can be seen to result in a 24 mm valvular area. Since the unloading/deflation curve represents the anatomy in the condition that the prosthesis will be subjected to, a prosthetic valve based on the unloading curve should be selected. The dimensions are used for example only, but in this example a person might pick a valve size of 21 mm based on the loading/inflation curve, where the correct size to reduce migration, reduce paravalvular leakage and improve hemodynamics would be a 24 mm valve.

It should be noted that the unloading/deflation curve of FIG. 9B can be attained during contraction of the balloon as shown, or the same data could be obtained if the balloon was completely contracted, and re-inflated shortly thereafter as may be required to not occlude a vessel for a longer period of time than desired.

The measurements for the dimensions of the expandable member 110 (and hence the lumen) may be taken by using an ultrasonic imaging head as discussed previously. However, a particularly preferred method for taking the measurements for the dimensions of the expandable member 110 (and hence the lumen) may be done by optical coherence tomography (OCT).

A fundamental aspect of OCT is the use of low coherence interferometry (either in the time domain or the Fourier domain). In conventional laser interferometry, the interference of light occurs over a distance of meters. In OCT, the use of broadband light sources (i.e., light sources that can emit light over a broad range of frequencies) enables the interference to be generated within a distance of micrometers. Such broadband light sources include super luminescent diodes (i.e., super bright light emitting diodes (LEDs)), extremely short pulsed lasers (i.e., femto-second lasers) and wavelength/frequency-swept lasers. White light can also be used as a broadband source.

Essentially, the combination of backscattered light from the sample arm and reference light from the reference arm gives rise to an interference pattern, but only if light from both arms have traveled “substantially the same” optical distance (where “substantially the same” indicates a difference of less than a coherence length). By scanning the mirror in the reference arm, a reflectivity profile of the sample can be obtained. Areas of the sample that reflect more light will create greater interference than areas that reflect less light. Any light that is outside the short coherence length will not contribute significantly to the interference signal. This reflectivity profile contains information about the spatial dimensions and location of structures within the sample. An OCT image (i.e., a cross-sectional tomograph), may be achieved by laterally combining multiple adjacent axial scans at different transverse positions.

OCT is particularly desirable for in vivo imaging since it can provide tomographic images of sub-surface biological structure with a few microns resolution. It is analogous to ultrasound imaging in that two-dimensional images of structure are built up from sequential adjacent longitudinal scans of backscatter versus depth into the tissue. However, in OCT, the probing radiation is light rather than sound waves, thus higher resolution measurements are possible.

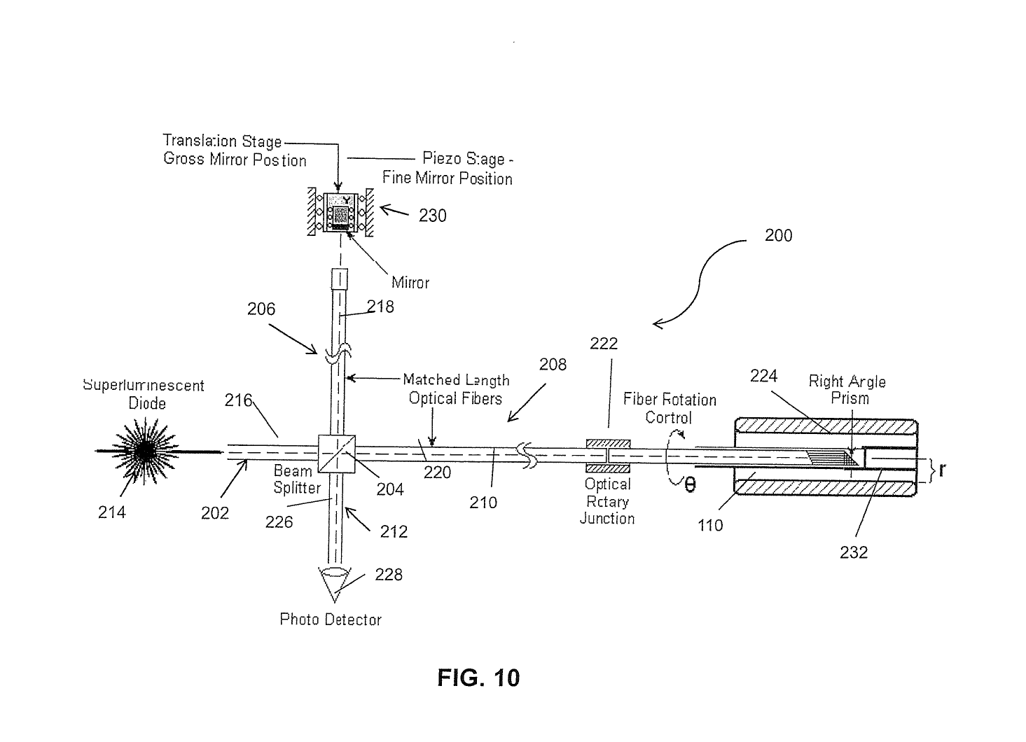

Referring to FIG. 10, there is shown a schematic view of an OCT imaging system 200 that makes use of the apparatus of the present invention. The OCT imaging system 200 includes five main parts: an illumination arm 202, a beam splitter/combiner 204, a reference arm 206, a sample arm 208 that incorporates a catheter 210 for insertion into a patient's body, and a detection arm 212. The OCT imaging system 200 is a basic schematic diagram for purposes of illustration and not limitation. Alternate OCT system configurations can be constructed.

The illumination arm 202 consists of a low coherence, broad band light source 214 which in an exemplary embodiment is a superluminescent diode. An optical fiber 216 transmits illumination light from light source 214 to the beam splitter/combiner 204. The illumination light is divided by splitter/combiner 204 into two beams: one beam is transmitted to optical fiber 218 of the reference arm 206 and the other beam is transmitted to optical fiber 220 in catheter 210 of the sample arm 208.

Reference arm 206 includes, in an exemplary embodiment, a two stage reference mirror 230 with coarse and fine position controls that can be used to match the optical path length to that of the optical fiber 220 in the catheter 210 of the sample arm 208. In another exemplary embodiment, there may be a single stage reference mirror capable of moving over longer distances than conventional reference mirrors. When the reference mirror moves, the image plane is changed.

The optical fiber 220 in catheter 210 delivers light to the site of interest within the patient's body. The optical fiber 220 is outfitted with a rotary optical junction 222 and terminates at a GRIN lens and right angle prism 224 inside an optically clear tubing 232 that can direct the light onto the wall of the expandable member. Inside the expandable member will be the inflation medium. The beam scanning can be performed radially, perpendicular to the longitudinal axis of catheter 210, or linearly along the catheter axis. The reflected/backscattered light from the site of interest is collected in the optical fiber 220 and delivered back to beam splitter/combiner 204. The sample beam from optical fiber 220 and the reference beam from optical fiber 218 are combined at beam splitter/combiner 204. The combined beam is then transmitted via optical fiber 226 in detection arm 212 to photodetector 228. The signal from the photodetector 228 may be used to tune the position of the reference mirror to establish “r,” the distance from the prism 224 to the wall of the expandable member 110 at any angular position of the optical fiber 220.

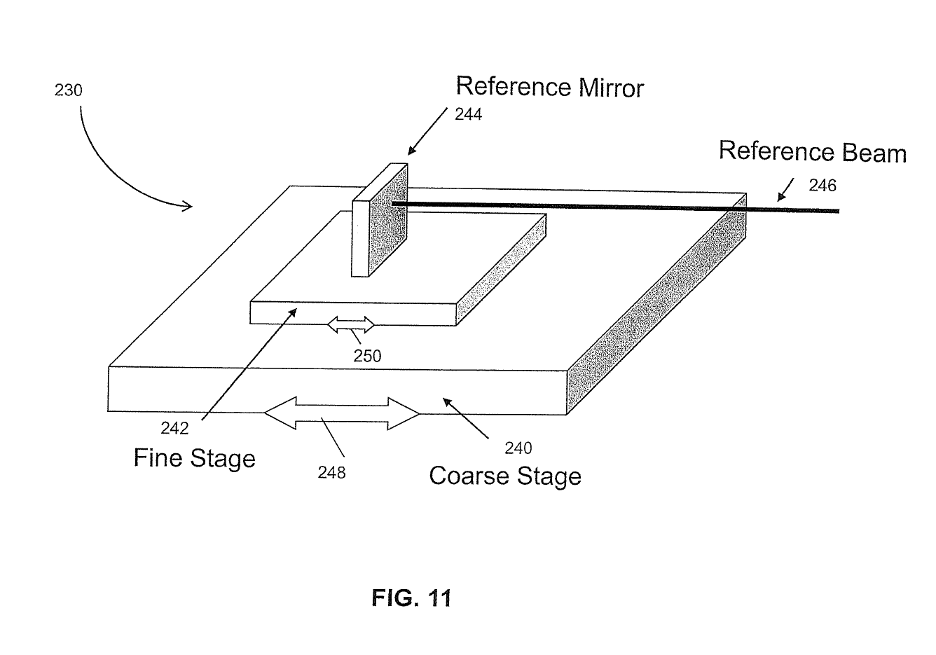

Referring now to FIG. 11, there is shown an enlarged perspective view of an exemplary embodiment of a dual stage mirror 230 which includes a coarse stage 240, a fine stage 242 and reference mirror 244 mounted on the fine stage 242. The coarse stage 240 has a movement in the range of about 0 to 20 millimeters (mm) and moves relatively slowly while the fine stage 242 has a movement in the range of +/−2 mm and moves very rapidly. It should be understood that the foregoing ranges of motion are for purposes of illustration and not limitation. The direction and magnitude of movement of the coarse stage 240 is indicated by double-ended arrow 248 while the direction and magnitude of movement of the fine stage 242 is indicated by double-ended arrow 250. The coarse stage 240 and the fine stage 242 may be translated by any device capable of linear motion including, but not limited to, ultrasonic actuators, piezoelectric actuators, voice coil actuators, electro-mechanical actuators and linear motor actuators.

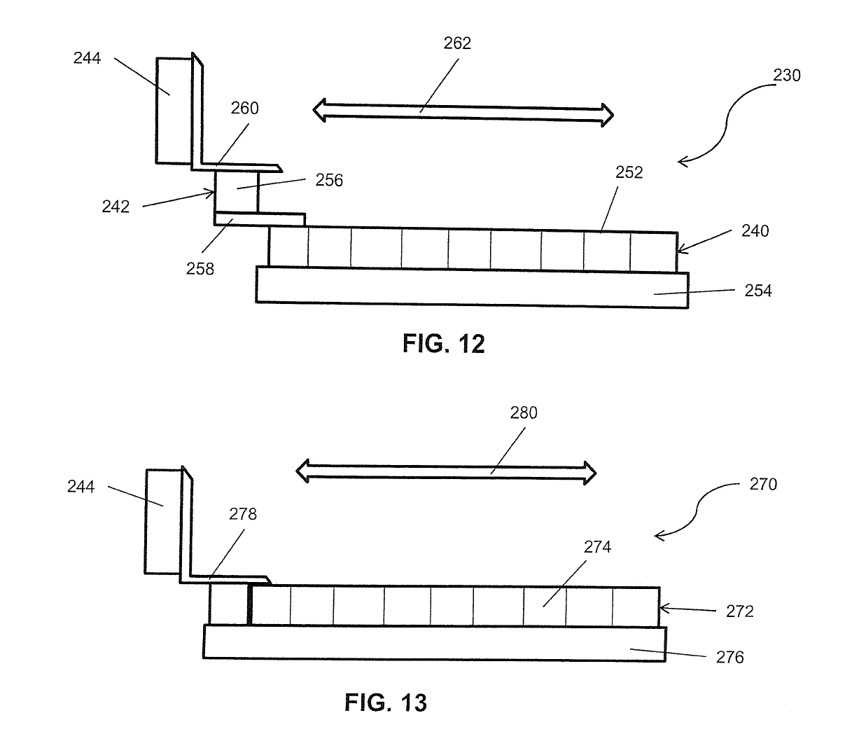

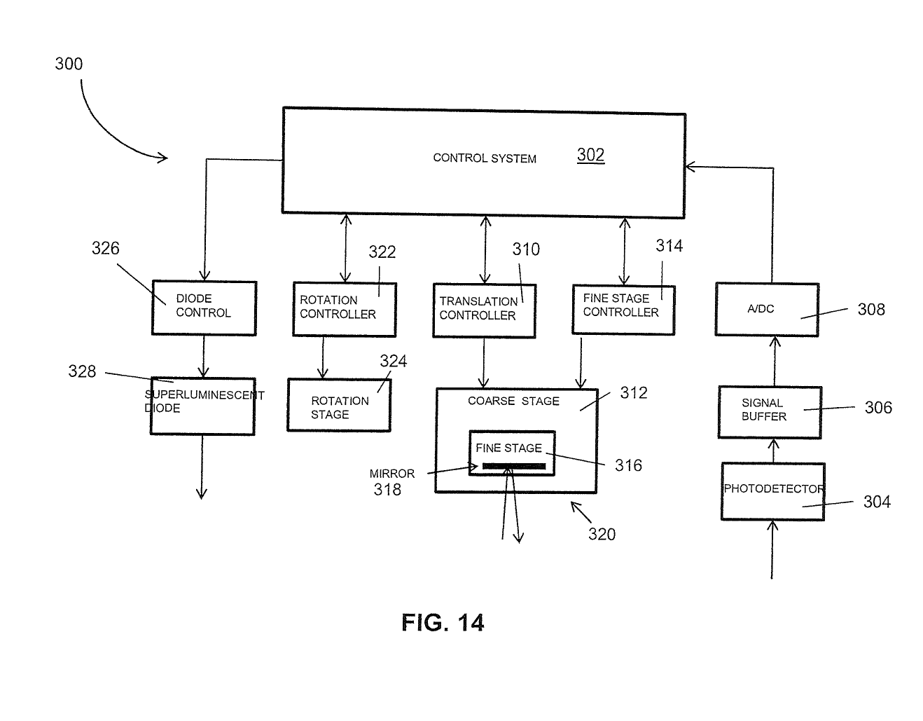

In one exemplary embodiment, the dual stage mirror 230 of FIG. 11 is illustrated in more detail in FIG. 12. The coarse stage 240 and fine stage 242 are translated by a piezoelectric actuator comprised of one or more piezoelectric crystals. That is, coarse stage 240 includes multiple piezoelectric crystals 252, each of which is capable of a small amount, such as 2 mm, of movement. With the multiple piezoelectric crystals 252 shown in FIG. 12, the coarse stage 240 is capable of a larger amount, such as 18 mm, of movement which is the cumulative movement of multiple crystals. The multiple piezoelectric crystals 252 sit on base 254. Fine stage 242 includes at least one piezoelectric crystal 256 which is capable of short, fast movement. Piezoelectric crystal 256 is situated on support 258 which is turn is supported by the coarse stage 240. Reference mirror 244 is attached to fine stage 242 by support 260. The movement of the coarse stage 240 and the fine stage 242 moves the reference mirror 244 with respect to the reference beam 246 to thereby obtain a reflectivity profile from the sample arm 208. The combined movement of the coarse stage 240 and fine stage 242 is indicated by double-ended arrow 262, which is a combination of double-ended arrows 248, 250 in FIG. 11. The movement of the coarse stage 240 and fine stage 242 enhances the field of view of the sample arm.

It should be understood that only an exemplary embodiment of the dual stage mirror has been shown and that other means of translating the reference mirror 244, including the actuators mentioned above, are included within the scope of the present invention. It should also be understood that while the dual stage mirror is capable of movement in the range of 0 to 20 mm, movement more or less than 20 mm, but more than that of a single stage mirror alone, is included within the scope of the present invention.

Referring to both FIGS. 10 and 11, the right angle prism 224 is moved to a desired location within the expandable member 110. The coarse stage 240 is moved over a large range such as 0 to 20 mm to do a scan. The photodetector 228 may pick up several peaks during the scan. The first peak may come from the interface of the optically clear tubing 232 and the inflation medium. The optically clear tubing 232 may have a radius of about 1 mm so the first peak will be at this distance of about 1 mm. The next peak may come from the interface of the inflation medium and expandable member 110. The expandable member 110 may have a radius of about 7 mm when slightly inflated so the second peak may be at this distance. Once the initial location of the inflation medium/expandable member interface is obtained, the coarse stage 240 can be moved accordingly, for example, to about 7 mm. With the fine stage 242 scanning over a range of 4 mm (+/−2 mm), any peaks between 5 to 9 mm can be picked up rapidly. The prism 224 can then be rotated a predetermined amount of degrees by optical rotary junction and the distance between the expandable member 110 and the prism 224 can be determined at this orientation. If the inflation medium/balloon interface is within the scanning range of +/−2 mm of the fine stage 242, it is only necessary to vibrate the fine stage to obtain the location of the interface at the angular orientation of interest. If the inflation medium/balloon interface is not within the scanning range of +/−2 mm of the fine stage 242, the coarse stage 240 may have to be translated to get the fine stage 242 back in range. The process is continued until a 360 degree scan has been completed of the expandable member 110. By determining the distance of the expandable member 110 from the prism 224, the distance of the lumen or hollow portion of the organ can be determined since the expandable member is situated against the lumen or hollow portion of the organ. As the expandable member 110 is expanded further to expand the lumen or hollow portion of the organ, for example in a valvuloplasty procedure, the coarse stage 240 may be needed to translate to find the expandable member 110 and put the fine stage 242 back in range.

In an exemplary embodiment, the dual stage mirror 230 may be replaced with a single stage mirror 270 shown in FIG. 13. The single stage 272 is translated by a piezoelectric actuator comprised of a plurality of piezoelectric crystals. The single stage 272 includes multiple piezoelectric crystals 274, each of which is capable of about 2 mm of movement. With the multiple piezoelectric crystals 274 shown in FIG. 13, the single stage 272 is capable of 20 mm of movement. The multiple piezoelectric crystals 274 sit on base 276. Reference mirror 244 is attached to the single stage 272 by support 278. The movement of the single stage 272, indicated by double-ended arrow 280, moves the reference mirror 244 with respect to the reference beam 246 to thereby obtain a reflectivity profile from the sample arm 208. The large movement of the single stage 272 enhances the field of focus of the sample arm. A dual stage mirror may be preferred because of rapid response time.

It should be understood that only an exemplary embodiment of the single stage mirror has been shown and that other means of translating the reference mirror 244, including the actuators mentioned above, are included within the scope of the present invention. It should also be understood that while the single stage mirror is capable of movement in the range of 0 to 20 mm, movement more or less than 20 mm, but more than that of a single stage mirror alone, is included within the scope of the present invention.

To maximize the imaging speed, a feedback loop is used to control the movement of the coarse stage, while the fine stage keeps oscillating rapidly. In the single stage embodiment, the single stage will scan over a short range based on the positions of the expandable member found in the neighboring locations on the expandable member. For example, if the position of neighboring member is at 7 m, the single stage scans from 5 to 9 mm An exemplary embodiment of a control apparatus 300 is shown in FIG. 12. Input from the photodetector 304 passes through signal buffer 306 and analog to digital converter (A/DC) 308 to control system 302. Control system 302 has suitable processors, memory and storage to be able to process the incoming signals and control the various parts of the control apparatus 300. Responsive to receiving the processed signal from the photodetector 304, the control system 302 may cause the translation controller 310 to cause coarse stage 312 of dual stage mirror 320 to move. Fine stage controller 314 controls fine stage 316 of dual stage mirror 320. The fine stage resonants periodically without active control. In an exemplary embodiment, the vibration frequency and distance may be reset in some situation using the final stage controller 314. For example, the fine stage 316 can be set to scan over 1 mm at a frequency of 100 Hz or 5 mm at 10 Hz. Mounted on fine stage 316 is a reference mirror 318.

Once the right angle prism 224 is through scanning a particular location of the expandable member 110, rotation controller 322 causes rotation stage 324 to rotate. The rotation stage is connected to the rotation controller and drives imaging probe via a rotary junction. The control system 302 may also control diode control 326 which controls the output of the superluminescent diode 328. The superluminescent diode 328 provides the light output for OCT imaging system 200.

There are numerous features of the exemplary embodiments from the above description of the OCT imaging system and feedback control system. With the present apparatus, OCT imaging over a long distance, such as 0 to 20 mm (and at the very least greater than the 0 to 2 mm range of conventional OCT apparatus), is possible. The OCT imaging system watches and tracks the movement of the expandable member. The OCT imaging system watches the movement of the expandable member and not the lumen or hollow portion of an organ. Moreover, the OCT imaging system may actually output dimensions of the expandable member, and hence also the lumen and hollow portion of an organ, which is very useful when determining the correct size for a replacement heart valve or other medical device. Additionally, the OCT imaging system may be calibrated by using the known distance of the optically clear tubing which then enables the OCT imaging system to accurately determine dimensions of other objects such as the expandable member as it expands and contracts. Lastly, the OCT imaging system may be used to determine compliance.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. An apparatus for expansion of an expandable member within a lumen or hollow portion of an organ comprising:
a force application device comprising an expandable member;
an assessment mechanism to determine assessment information comprising a physical property of the expandable member and a force applied by the expandable member at multiple points in time as the expandable member applies force to the lumen or hollow portion of the organ; and
an instrumentality for receiving the assessment information and based on the assessment information, analyzing a change of the lumen or hollow portion of the organ as the expandable member applies force to the lumen or hollow portion of the organ.

2. The apparatus of claim 1 wherein the expandable member applies force upon expansion and/or contraction of the expandable member.

3. The apparatus of claim 1 wherein a change of the lumen or hollow portion of the organ is a rate of change of the lumen or hollow portion of the organ.

4. The apparatus of claim 1 wherein the physical property is a dimension of the expandable member.

5. The apparatus of claim 1 wherein the physical property at multiple points in time defines the perimeter of a lumen.

6. The apparatus of claim 1 wherein the change of the lumen or hollow portion of the organ is a compliance of the lumen or hollow portion of the organ wherein compliance is defined by the following equation:
$$compliance = \frac{θ - {θ}^{′}}{F 1 - F 2}$$
wherein
θ=a physical property at a location of the expandable member at a first point in time;
θ′=a physical property at the same location of the expandable member at a second point in time;
F1=a force applied by the expandable member at the first point in time; and
F2=a force applied by the expandable member at the second point in time.

7. The apparatus of claim 6 wherein F1 is a pressure applied to the expandable member at the first point in time and F2 is a pressure applied to the expandable member at the second point in time.

8. The apparatus of claim 1 wherein the assessment mechanism is retracted axially to provide a third dimension of physical property data.

9. The apparatus of claim 1 further comprising expansion of the expandable member and adjusting the expanding of the expandable member according to the change of the lumen or hollow portion of the organ.

10. The apparatus of claim 1 wherein the assessment information includes a force applied to the expandable member at multiple points in time as the expandable member is further expanded against the lumen or hollow portion of the organ to expand the lumen or hollow portion of the organ beyond an unexpanded state of the lumen or hollow portion of the organ.

11. The apparatus of claim 1 wherein a valvuloplasty procedure is performed as the expandable member is being expanded against the lumen or hollow portion of the organ.

12. The apparatus of claim 1 wherein the instrumentality is a data processing unit.

13. The apparatus of claim 1 further comprising a medium for expanding the expandable member.

14. The apparatus of claim 13 wherein the medium is selected from the group consisting of saline, acoustic gel, dielectric fluid, blood, gas and contrast medium.

15. The apparatus of claim 13 wherein the medium is a liquid or a gas.

16. The apparatus of claim 1 wherein the expandable member is a balloon.

17. The apparatus of claim 1 wherein the assessment mechanism comprises an imaging device to view the expandable member during expansion of the expandable member.

18. The apparatus of claim 17 wherein the imaging device is an optical imaging device.

19. The apparatus of claim 1 wherein the assessment mechanism is optical coherence tomography.

20. The apparatus of claim 17 wherein the imaging device is an ultrasound imaging device.

21. The apparatus of claim 1 wherein the assessment mechanism comprises a pressure sensor to measure the pressure applied to the expandable member.

22. An optical coherence tomography apparatus comprising:
an illumination arm for transmitting illumination light;
a reference arm;
a sample arm;
a beam splitter for splitting the illumination light for transmission to the reference arm and sample arm;
a long stage mirror moving over a distance between 0 and greater than 2 mm; and
a photodetector for receiving the light reflected from the sample arm and reference arm.

23. The apparatus of claim 22 wherein the long stage mirror is a single stage mirror focusing over a distance between 0 and greater than 10 mm.

24. The apparatus of claim 22 wherein the long stage mirror is a dual stage mirror comprising a coarse stage, a fine stage and a mirror, the coarse stage moving the mirror over a long distance while the fine stage moving the mirror over a short distance.

25. The apparatus of claim 24 further comprising a control apparatus for controlling the movement of the dual stage mirror.

26. The apparatus of claim 22 wherein the control apparatus comprises a feedback loop for controlling the movement of the dual stage mirror, the feedback loop comprising a signal receiver to receive a signal from the photodetector and input the signal to a control system, the control system receiving the signal, processing it and outputting a signal to a translation controller, the translation controller controlling the movement of the coarse translation stage.

27. The apparatus of claim 22 wherein the sample arm comprises an expandable member inserted into a human patient.

28. The apparatus of claim 25 wherein the apparatus tracks a movement of the expandable member.

29. The apparatus of claim 27 wherein the apparatus watches the expandable member and not a lumen or hollow portion of an organ.

30. The apparatus of claim 22 wherein the apparatus measures a physical dimension.

31. The apparatus of claim 27 wherein the apparatus measures a radius of the expandable member at multiple points around a circumference of the expandable member.

32. The apparatus of claim 28 wherein the apparatus determines compliance of a lumen or hollow portion of an organ.

33. The apparatus of claim 27 further comprising an optically clear tubing having a known dimension within the expandable member, the apparatus being calibrated by using the known dimension of the optically clear tubing and using that known dimension to determine another dimension.

34. The apparatus of claim 27 further comprising an optically clear tubing having a known dimension within the expandable member, the apparatus being calibrated by using the known dimension of the optically clear tubing and using that known dimension to determine a radius of the expandable member.

35. An optical coherence tomography apparatus comprising:
an illumination arm for transmitting illumination light;
a reference arm having a long stage mirror moving over a distance between 0 and greater than 2 mm;
a sample arm having a catheter and, at a distal end of the catheter, an expandable member;
a lens within the expandable member to receive the illumination light and illuminate an inside surface of the expandable member;
a beam splitter for splitting the illumination light for transmission to the reference arm and sample arm; and
a photodetector for receiving the light reflected from the lens in the sample arm and the mirror in the reference arm and determining a dimension.

36. The apparatus of claim 35 wherein the long stage mirror is a single stage mirror moving over a distance between 0 and greater than 10 mm.

37. The apparatus of claim 35 wherein the long stage mirror is a dual stage mirror comprising a coarse stage, a fine stage and a mirror, the coarse stage moving the mirror over a long distance while the fine stage moving the mirror over a short distance.

38. The apparatus of claim 37 further comprising a feedback loop for controlling the movement of the dual stage mirror, the feedback loop comprising a signal receiver to receive a signal from the photodetector and input the signal to a control system, the control system receiving the signal, processing it and outputting a signal to a translation controller, the translation controller controlling the movement of the coarse translation stage.

39. The apparatus of claim 35 wherein the dimension is a dimension of the expandable member.

40. The apparatus of claim 35 wherein the dimension of the expandable member is a radius of the expandable member.

41. The apparatus of claim 35 wherein the dimension is a compliance of a lumen or hollow portion of an organ.

42. A method for expansion of an expandable member within a lumen or hollow portion of an organ comprising:
expanding the expandable member applying a force to a lumen or hollow portion of an organ;
determining a physical property of the expandable member and the force of the expandable member as the expandable member is being expanded against the lumen or hollow portion of the organ; and
determining a change of the lumen or hollow portion of the organ as the expandable member is being changed.

43. The method of claim 42 wherein a change of the lumen or hollow portion of the organ is a rate of change of the lumen or hollow portion of the organ.

44. The method of claim 42 wherein the physical property is a dimension of the expandable member.

45. The method of claim 42 wherein the physical property is a dimension of the expandable member and wherein the change of the lumen or hollow portion of the organ is a compliance of the lumen or hollow portion of the organ wherein compliance is defined by the following equation:
$$compliance = \frac{θ - {θ}^{′}}{F 1 - F 2}$$
wherein
θ=a physical property at a location of the expandable member at a first point in time;
θ′=a physical property at the same location of the expandable member at a second point in time;
F1=a force applied by the expandable member at the first point in time; and
F2=a force applied by the expandable member at the second point in time.

46. The method of claim 45 wherein F1 is a pressure applied to the expandable member at the first point in time and F2 is a pressure applied to the expandable member at the second point in time.

47. The method of claim 42 further comprising adjusting the expanding of the expandable member according to the change of the lumen or hollow portion of the organ.

48. The method of claim 42 wherein determining a physical property includes a force applied to the expandable member as the expandable member is further expanded against the lumen or hollow portion of the organ to expand the lumen or hollow portion of the organ beyond an unexpanded state of the lumen or hollow portion of the organ.

49. The method of claim 42 wherein a valvuloplasty procedure is performed as the expandable member is being expanded against the lumen or hollow portion of the organ.

50. The method of claim 42 wherein the physical property is measured at multiple locations around a circumference of the expandable member.

51. The method of claim 42 wherein the physical property is determined by an imaging device to view the expandable member during expansion of the expandable member.

52. The method of claim 51 wherein the imaging device is an optical coherence tomography device.

53. The method of claim 42 wherein the force is applied at a constant rate versus time.

54. The method of claim 42 wherein the force is applied by pulsing the force for a period of time.

55. The method of claim 53 wherein the pulsing is done repeatedly.

56. The method of claim 42 wherein the lumen is a cardiac valve, atrial appendage, coronary lumen, peripheral lumen, abdominal lumen, biliary duct or fallopian tube.

57. The method of claim 42 wherein the expansion is of a lumen and the physical property is a dimension of the expandable member and further comprising:
choosing a medical device for inserting into the lumen according to a determined dimension of the expandable member; and
orienting the medical device according to the determined dimension of the expandable member.

58. A method for expansion of an expandable member within a lumen or hollow portion of an organ comprising:
deploying an expandable member within the body of a patient;
expanding the expandable member by applying a force by the expandable member;
determining a dimension of the expandable member and the force of the expandable member at multiple points in time as the expandable member is being expanded against the lumen or hollow portion of the organ;
determining a compliance of the lumen or hollow portion of the organ as the expandable member is being expanded against the lumen or hollow portion of the organ, wherein the compliance is a rate of change of the outer dimension with a change of force of the expandable member; and
adjusting a rate of the expanding of the expandable member according to the compliance of the lumen or hollow portion of the organ.

59. The method of claim 58 wherein compliance is defined by the following equation:
$$compliance = \frac{θ - {θ}^{′}}{F 1 - F 2}$$
wherein
θ=a physical property at a location of the expandable member at a first point in time;
θ′=a physical property at the same location of the expandable member at a second point in time;
F1=a force applied by the expandable member at the first point in time; and
F2=a force applied by the expandable member at the second point in time.

60. The method of claim 58 wherein the dimension is measured at multiple locations around a circumference of the expandable member.

61. The method of claim 58 wherein the dimensions are determined by an imaging device to view the expandable member during expansion of the expandable member.

62. The method of claim 61 wherein the imaging device is an optical coherence tomography device.

63. The method of claim 58 wherein the pressure is applied at a constant rate versus time.

64. The method of claim 58 wherein the pressure is applied by pulsing the pressure for a period of time.

65. The method of claim 64 wherein the pulsing is done repeatedly.

66. The method of claim 58 wherein the lumen is a cardiac valve, atrial appendage, coronary lumen, peripheral lumen, abdominal lumen, biliary duct or fallopian tube.

67. The method of claim 58 wherein the expansion is of a lumen and further comprising:
choosing a medical device for inserting into the lumen according to a determined dimension of the expandable member.

68. The method of claim 58 wherein the expansion is of a lumen and further comprising:
orienting the medical device according to the determined dimension of the expandable member.

69. A method to determine a dimension of an expandable member with the aid of an optical coherence tomography apparatus comprising a reference arm having a long stage mirror and a sample arm, the method comprising:
deploying an expandable member within the body of a patient, the expandable member forming a part of the sample arm of the optical coherence tomography apparatus;
expanding the expandable member by applying a force by the expandable member;
determining a dimension of the expandable member by scanning the long stage mirror during the expanding of the expandable member to keep the expandable member in the image plane of the long stage mirror.

70. The method of claim 69 wherein the long stage mirror scanning over a distance between 0 and greater than 2 millimeters.

71. The method of claim 69 wherein the long stage mirror scanning over a distance between 0 and greater than 10 millimeters.

72. The method of claim 69 further comprising:
choosing a medical device for inserting into the body of the patient according to a determined dimension of the expandable member; and
orienting the medical device according to the determined dimension of the expandable member.

73. A method of treating the body of a patient comprising:
inserting a force application device comprising an expandable member and an assessment mechanism into the body of the patient;
expanding the expandable member;
obtaining information pertaining to a dimension of the expandable member from the assessment mechanism; and
analyzing the information obtained from the assessment mechanism to determine a dimension of the expandable member.
